(19) United States
(12) Patent Application Publication
(10) Pub. No.: US 2005/0169873 A1
(43) Pub. Date: Aug. 4, 2005

(54) Styling composition comprising, in a predominantly aqueous medium, an elastic cationic polyurethane, processes using it and uses thereof

(76) Inventors: Isabelle Rollat, Paris (FR); Pascale Cothias, Montigny Le Bretonneux (FR); Katarina Benabdillah, Le Plessis Bouchard (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/014,706

(22) Filed: Dec. 20, 2004

Related U.S. Application Data
(60) Provisional application No. 60/562,555, filed on Apr. 16, 2004.

(30) Foreign Application Priority Data
Dec. 19, 2003 (FR) 0315107

(51) Int. Cl. A61K 7/06; A61K 7/11

(52) U.S. Cl. 424/70.17

(57) ABSTRACT

Disclosed herein is a styling cosmetic composition comprising, in a cosmetically acceptable medium predominantly comprising water, at least one cationic polyurethane of elastic nature. Also disclosed are cosmetic treatment processes for shaping or holding the hairstyle comprising applying the styling cosmetic composition to the hair. There are also disclosed methods of shaping or holding the hair comprising applying the composition to the hair, such that the resulting hair exhibits at least one property chosen from persistence over time and resistant to moisture.

This application claims benefit under 35 U.S.C. §119 of U.S. Provisional Application No. 60/562,555, filed Apr. 16, 2004 and which is hereby incorporated by reference.

Disclosed herein are styling cosmetic compositions comprising, in a cosmetically acceptable medium predominantly comprising water, at least one elastic cationic polyurethane. Further disclosed herein are a process for shaping or holding the hairstyle in which this composition is used and uses of this composition.

The cosmetic compositions for shaping and/or holding the hairstyle that are the most widely available on the cosmetics market are spray compositions consisting essentially of a solution, which may be alcoholic, and of at least one components, defined herein as fixing components, which may be polymer resins, the function of which is to form welds between the hairs. These fixing components may be formulated as a mixture with various cosmetic adjuvants. This composition may be packaged either in a suitable aerosol container pressurized using a propellant, or in a pump-dispenser bottle.

Aerosol systems for fixing the hair may contain a liquid phase (or fluid) and a propellant. The liquid phase may contain the fixing components and a suitable solvent.

Once applied to the hair, the liquid phase dries, which can allow the formation of welds required for fixing the hair by the fixing components. The welds should be rigid enough to hold the hairs, and to do so with a sufficient persistence of the effects, including with respect to moisture. However, they should also be fragile enough for the user to be able, by combing or brushing the hair, to destroy them without hurting the scalp or damaging the hair. The compositions must also be stable over time. A good cosmetic effect on the hair is also sought, such as improved softness and disentangling properties.

It is moreover sought to reduce the amount of volatile solvents present in these compositions, for environmental reasons. The total or partial replacement of volatile solvents such as alcohol with water can be reflected by a dramatic reduction in the fixing properties, the persistence of the styling effect over time and the cosmetic properties.

French patent application FR 2 815 350 discloses styling compositions comprising cationic polyurethanes of elastic nature in a predominantly alcoholic medium.

The present inventors have surprisingly discovered that these cationic polyurethanes of elastic nature may be used to prepare predominantly aqueous styling compositions that are stable for several months and that allow good fixing and good hold of the hair, i.e. a styling effect that persists throughout the day, or even for several days, with good moisture resistance, and that is easy to eliminate by shampooing. These compositions can also make it possible to give the hair good cosmetic properties, such as softness or disentangling.

Furthermore, their predominantly aqueous base can make the disclosed compositions ecologically advantageous.

Disclosed herein are styling cosmetic compositions comprising, in a cosmetically acceptable medium predominantly comprising water, at least one cationic polyurethane of elastic nature.

Further disclosed herein is a cosmetic treatment process for shaping or holding the hairstyle, comprising the use of the disclosed cosmetic composition.

Further disclosed herein are the uses of cationic polyurethanes of elastic nature in predominantly aqueous cosmetic compositions, to obtain fixing of the hair that persists over time and/or good moisture resistance and/or good cosmeticity.

Other embodiments disclosed herein will emerge even more clearly on reading the description and the examples that follow.

As defined herein, the term “styling composition” or “styling cosmetic composition” means a composition for shaping or holding the hairstyle.

As defined herein, a cosmetically acceptable medium is a medium predominantly comprising water and optionally at least one organic solvent.

As defined herein, the term “medium predominantly comprising water” means a medium comprising more than 50%, such as more than 70% and further such as more than 85% by weight of water relative to the total weight of the composition. This medium should contain less than 99.9% by weight of water relative to the total weight of the composition.

As defined herein, the term “organic solvent” means an organic compound with a molecular weight of less than about 500, which is liquid at a temperature of about 25° C. and at atmospheric pressure. The organic compound, for example, may be polar.

For example, this organic solvent may be an alcohol. This alcohol, for example, can be chosen from C₁-C₄ lower alcohols, for example ethanol, isopropanol, tert-butanol or n-butanol; polyols, for example propylene glycol, and polyol ethers, and mixtures thereof.

In certain embodiment, the compositions disclosed herein, for example, may contain no C₁-C₄ alcohol, and may contain no organic solvent.

The polyurethanes used in the compositions disclosed herein are fixing polyurethanes, forming non-tacky, non-brittle films and are capable of plastic and elastic deformations.

As defined herein, the term “fixing polyurethane” means a polyurethane whose function is to give to or maintain on the hairstyle a given shape.

As defined herein, the term “elastic” means a compound that has the property of totally or partially regaining its shape and/or its volume after having been deformed.

The cationic polyurethanes of elastic nature used in the compositions disclosed herein are, for example, the polyurethanes described in French patent application FR 2 815 350. The part of the patent application that is devoted to the description of polyurethanes and to their synthesis is incorporated by reference into the present patent application.

The cationic polyurethanes of elastic nature may be comprised of the following types of units:
(a1) cationic units derived from at least one tertiary or quaternary amine comprising at least two reactive functions containing labile hydrogen,
(a2) nonionic units derived from nonionic polymers bearing at their ends reactive functions containing labile hydrogen and having a glass transition temperature (Tg), measured by differential thermal analysis, of less than 10° C.,
(a3) optionally, nonionic units derived from nonionic monomer compounds comprising at least two functions containing labile hydrogen, and
(b) units derived from at least one diisocyanate.

The tertiary or quaternary amines forming the cationic units (a1) may, for example, be chosen from the compounds corresponding to one of the following formulae: 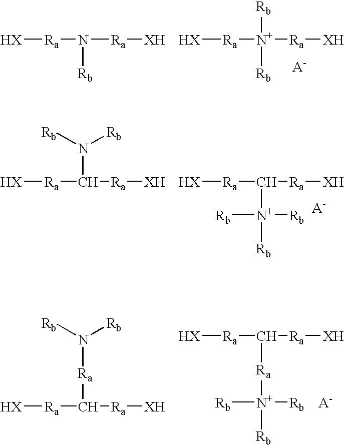
wherein
each Rₐ is independently chosen from linear and branched C₁₋₆ alkylene groups, C₃₋₆ cycloalkylene groups and arylene groups, optionally substituted with at least one halogen atom, wherein each Rₐ may comprise at least one hetero atom chosen from O, N, P and S;
each R_{b} is independently chosen from C₁₋₆ alkyls, C₃₋₆ cycloalkyls and aryl groups, optionally substituted with at least one halogen atom, wherein each R_{b} may comprise at least one hetero atom chosen from O, N, P and S;
X is independently chosen from at least one oxygen atom, sulfur atom, an NH and a NR_{c} group, in which R_{c} represents a C₁₋₆ alkyl group; and
A—is a physiologically acceptable counterion.

The tertiary amines forming the cationic units of (a1) may be chosen, for example, from N-methyldiethanolamine and N-tert-butyldiethanolamine.

The tertiary and quaternary amines forming the cationic units (a1) of the polyurethanes may also be chosen from, for example, polymers comprising tertiary and quaternary amine functions, bearing at their ends at least one reactive function containing labile hydrogen. The weight-average molar mass of these polymers comprising tertiary and quaternary amine functions may, for example, range from 400 to 10 000.

Examples of such polymers comprising amine functions include, for example, the polyesters derived from the polycondensation of N-methyldiethanolamine and of adipic acid.

When the amines forming the cationic units (a1) are compounds comprising tertiary amine function(s), at least one of these amine functions may be neutralized with a suitable neutralizer such as a physiologically acceptable organic or mineral acid. Physiologically acceptable organic or mineral acids are chosen from, for example, hydrochloric acid and acetic acid.

The second type of units forming the polyurethane compositions disclosed herein are macromolecular units, known as units (a2), derived from nonionic polymers bearing at their ends reactive functions containing labile hydrogen and having a glass transition temperatures (Tg), measured by differential thermal analysis, of less than 10° C.

The viscoelastic properties of the polyurethane compositions disclosed herein may be advantageous when the units (a2) are polymer derivatives with a glass transition temperature, for example, of less than 0° C. and further, for example, of less than −10° C.

The polymer derivates of units (a2) can have a weight-average molar mass, for example, ranging from 400 to 10 000 and further, for example, ranging from 1000 to 5000.

The nonionic polymers capable of forming the nonionic units (a2) are chosen, for example, from ethers, polyesters, polysiloxanes, copolymers of ethylene and of butylene, polycarbonates and fluoro polymers.

Further the non-ionic polymers may be chosen from, for example, polyethers for example, poly(tetramethylene oxide).

The diisocyanate forming the units (b) may be chosen from aliphatic, alicyclic and aromatic diisocyanates, for example, methylenediphenyl diisocyanate, methylenecyclohexane diisocyanate, isophorone diisocyanate, toluene diisocyanate, naphthalene diisocyanate, butane diisocyanate and hexyl diisocyanate or mixtures thereof.

The cationic polyurethanes of elastic nature of the composition disclosed herein may further comprise units (a3) derived from nonionic monomer compounds comprising at least two functions containing labile hydrogen.

These units (a3) may be derived from monomer compounds chosen from, for example, neopentyl glycol, hexaethylene glycol and aminoethanol.

A physical parameter characterizing the viscoelastic properties of the cationic polyurethanes disclosed herein may be their tensile recovery. The part of French Patent Application FR 2 815 350 that is devoted to this parameter and to its determination is incorporated by reference into the present patent application. The cationic polyurethanes of elastic nature in the composition disclosed herein have an instantaneous recovery (Ri), measured under the conditions of Patent Application FR 2 815 350, for example, comprising from 5% to 95%, comprising from 20% to 90% and comprising from 35% to 85%.

The glass transition temperature (Tg) of the nonionic polymers forming the units (a2) and of the cationic polyurethanes disclosed herein may be measured by differential thermal analysis (DSC, differential scanning calorimetry) according to ASTM standard D3418-97.

The cationic polyurethanes of elastic nature disclosed herein may have at least two glass transition temperatures, at least one of which is less than 10° C., less than 0° C. or less than −10° C., and at least one other is greater than or equal to room temperature (20° C.).

The instantaneous recovery, and consequently the viscoelastic properties of the polyurethanes disclosed herein, may depend on the fractions of the various monomer units (a1), (a2), (a3) and (b).

The fraction of units (a1) can be sufficient to give the polymer their positive charge responsible for their good affinity towards keratin substrates. The units of (a2) may be present in a weight fraction that can be sufficient for the polyurethanes to have at least one glass transition temperature of less than 10° C. and to not form brittle films.

In general, the units (a1) can range, for example, from 1% to 90% and further, for example, from 5% to 60% by weight, the units (a2) can range, for example, from 10% to 80% and further, for example, from 40% to 70% by weight, and the units (a3) can range, for example, from 0 to 50% and further, for example, from 0 to 30% by weight relative to the total weight of the polymers.

The units (b) may be present in an essentially stoichiometric amount relative to the sum of the units (a1), (a2) and (a3). Specifically, producing polyurethanes with high molar masses assumes a number of isocyanate functions that can be virtually identical to the number of functions containing labile hydrogen. A person skilled in the art will know how to select a possible molar excess of one or other type of function to adjust the molar mass to the desired value.

The cosmetic composition disclosed herein comprises the elastic cationic polyurethane(s) in an amount ranging from, for example, 0.1% to 20% by weight and further, for example, from 0.5% to 12% by weight relative to the total weight of the composition.

The fixing cosmetic composition disclosed herein may also contain at least one adjuvant chosen, for example, from other elastic cationic non-polyurethane fixing polymers (additional fixing polymers); silicones in soluble, dispersed and micro-dispersed form; thickening polymers; nonionic, anionic, cationic and amphoteric surfactants; ceramides and pseudoceramides; vitamins and provitamins including, for example, panthenol, plant, animal, mineral and synthetic oils; waxes other than ceramides and pseudoceramides; water-soluble and liposoluble, silicone and non-silicone sunscreens; glycerol; permanent and temporary dyes; nacreous agents and opacifiers; sequestering agents; plasticizers; solubilizers; acidifying agents; basifying agents; mineral thickeners; antioxidants; hydroxy acids; penetrating agents; fragrances; fragrance solubilizers (peptizers); preserving agents; anticorrosion agents; and treating active agents.

The at least one adjuvant may be present in the cosmetic composition disclosed herein in an amount ranging, for example, from 0 to 20% by weight relative to the total weight of the cosmetic composition.

A person skilled in the art will take care to select the at least one adjuvant(s) and the amount thereof such that they do not harm the properties of the compositions.

The compositions disclosed herein may further at least one additional cosmetic active agent chosen, for example, from additional fixing polymers other than elastic cationic polyurethanes, thickening polymers, surfactants and nacreous agents or opacifiers.

Thus, for example, the compositions disclosed herein may contain at least one additional fixing polymer other than the cationic polyurethanes of elastic nature, chosen, for example from the following lists.

Anionic polymers comprising carboxylic groups, for example:
A) acrylic and methacrylic acid homo- and copolymers, and salts thereof, such as the products sold under the names Versicol® E and K by the company Allied Colloid, and Ultrahold® by the company BASF; copolymers of acrylic acid and of acrylamide, and sodium salts of polyhydroxycarboxylic acids;
B) copolymers of acrylic and methacrylic acid with a monoethylenic monomer chosen from, for example, ethylene, styrene, vinyl esters, acrylic and methacrylic acid esters. These copolymers may be optionally grafted onto a polyalkylene glycol, for example, polyethylene glycol and optionally crosslinked. Such polymers are described, for example, in French Patent No. 1 222 944 and German Patent Application No. 2 330 956. In one embodiment, the copolymer may be chosen from copolymers whose chain comprises at least one optionally N-alkylated and/or hydroxyalkylated acrylamide unit in their chain as described, for example, in Luxembourg Patent Applications Nos. 75370 and 75371; copolymers of acrylic acid and of C₁-C₄ alkyl methacrylates and terpolymers of vinylpyrrolidone; of acrylic acid and of methacrylate of C₁-C₂₀ alkyl, for example of lauryl, such as the product sold by the company ISP under the name Acrylidone® LM; and methacrylic acid/ethyl acrylate/tert-butyl acrylate terpolymers such as the product sold under the name Luvimer® 100 P by the company BASF;
C) copolymers derived from crotonic acid, chosen from, for example, those comprising at least one vinyl acetate or propionate unit in their chain and optionally other monomers chosen from, for example, allylic esters and methallylic esters, vinyl ether and vinyl ester of a linear and branched saturated carboxylic acid with a long hydrocarbon-based chain, for example, those comprising at least 5 carbon atoms, optionally grafted and crosslinked, or alternatively another vinyl, allylic or methallylic ester monomer of an α- or β-cyclic carboxylic acid. Such polymers are described, for example, in French Patent Nos. 1 222 944, 1 580 545, 2 265 782, 2 265 781, 1 564 110 and 2 439 798. A commercial product chosen from this category includes the resins 28-29-30 sold by the company National Starch;
D) copolymers derived from C₄-C₈ monounsaturated carboxylic acids and anhydrides chosen, for example, from:
copolymers comprising (i) at least one maleic, fumaric and itaconic acid and anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described, for example, in U.S. Pat. Nos. 2,047,398, 2,723,248 and 2,102,113 and GB Patent No. 839 805. Exemplary commercial products are those sold under the names Gantrez® AN or ES by the company ISP;
copolymers chosen, for example, from (i) at least one maleic, citraconic and itaconic anhydride unit and (ii) at least one monomer chosen from allylic and methallylic esters optionally comprising at least one group chosen from acrylamide, methacrylamide and α-olefin groups, acrylic and methacrylic esters, acrylic and methacrylic acids and vinylpyrrolidone in their chain, the anhydride functions of these copolymers optionally being monoesterified or monoamidated. These polymers are described, for example, in French Patent Nos. 2 350 384 and 2 357 241; and
E) polyacrylamides comprising at least one carboxylate group.

The polymers comprising at least one sulfonic group may, for example, be chosen from polymers comprising at least one unit chosen from vinylsulfonic, styrenesulfonic, naphthalenesulfonic and acrylamidoalkylsulfonic units.

The polymers comprising at least one sulfonic group may be chosen, for example, from:
polyvinylsulfonic acid salts having a molecular weight ranging from 1000 to 100 000, as well as the copolymers with at least one unsaturated comonomer chosen from acrylic and methacrylic acids and their esters, as well as acrylamide and its derivatives, vinyl ethers and vinylpyrrolidone;
polystyrenesulfonic acid salts, for example, the sodium salts sold for example under the name Flexane200 by National Starch. These compounds are described in French Patent No. FR 2 198 719;
polyacrylamidesulfonic acid salts, such as those described in U.S. Pat. No. 4,128,631, for example, polyacrylamidoethylpropanesulfonic acid.

As used herein, the anionic polymers may, for example, be chosen from acrylic acid copolymers such as the acrylic acid/ethyl acrylate/N-tert-butylacrylamide terpolymers sold under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold for example under the name Gantrez® by the company ISP, the copolymers of methacrylic acid and of methyl methacrylate sold under the name Eudragite® L by the company Rohm Pharma, the copolymers of methacrylic acid and of ethyl acrylate sold under the name Luvimer® MAEX or MAE by the company BASF and the vinyl acetate/crotonic acid copolymers and the vinyl acetate/crotonic acid copolymers grafted with polyethylene glycol sold under the name Aristoflex® A by the company BASF.

Among the previously described anionic fixing polymers, mention is made to those chosen from the methyl vinyl ether/monoesterified maleic anhydride copolymers sold under the name Gantrez® ES 425 by the company ISP, the acrylic acid/ethyl acrylate/N-tert-butylacrylamide terpolymers sold under the name Ultraholde Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer® MAEX or MAE by the company BASF and the vinylpyrrolidone/acrylic acid/lauryl methacrylate terpolymers sold under the name Acrylidone® LM by the company ISP.

The amphoteric fixing polymers may be chosen, for example, from polymers comprising units B and C distributed randomly in the polymer chain, wherein B is chosen from at least one unit derived from a monomer comprising at least one basic nitrogen atom and C is chosen from at least one unit derived from an acid monomer comprising at least one carboxylic or sulfonic group. Alternatively, B and C may be chosen from groups derived from carboxybetaine and sulfobetaine zwitterionic monomers;

B and C may also be chosen from cationic polymer chains comprising at least one primary, secondary, tertiary and quaternary amine group, wherein the at least one amine group bears a carboxylic or sulfonic group connected via a hydrocarbon-based group; or B and C may form part of a chain of a polymer comprising an α,β-dicarboxylic ethylene unit wherein at least one of the carboxylic groups has been reacted with a polyamine comprising at least one primary or secondary amine group.

The amphoteric fixing polymers as defined herein may be chosen from, for example:

(1) polymers resulting from the copolymerization of at least one monomer derived from a vinyl compound bearing at least one carboxylic group such as acrylic acid, methacrylic acid, maleic acid, a-chloroacrylic acid, and at least one basic monomer derived from a substituted vinyl compound comprising at least one basic atom, such as, dialkylaminoalkyl methacrylate and acrylate, dialkylaminoalkylmethacrylamides and -acrylamides. Such compounds are described in U.S. Pat. No. 3,836,537.

(2) polymers comprising units derived from:
a) at least one monomer chosen from acrylamides and methacrylamides substituted on the nitrogen atom with an alkyl group,
b) at least one acidic comonomer comprising at least one reactive carboxylic group, and
c) at least one basic comonomer such as esters comprising at least one substituent chosen from primary, secondary, tertiary and quaternary amine substituents of acrylic and methacrylic acids and the product of quaternization of dimethylaminoethyl methacrylate with dimethyl or diethyl sulfate.

The N-substituted acrylamides and methacrylamides which may be used in the compositions disclosed herein may be chosen from groups wherein the alkyl groups have from 2 to 12 carbon atoms, for example, N-ethylacrylamide, N-tert-butylacrylamide, N-tert-octylacrylamide, N-octylacrylamide, N-decylacrylamide, N-dodecylacrylamide and the corresponding methacrylamides.

The at least one acidic comonomer may be chosen from acrylic acids, methacrylic acids, crotonic acids, itaconic acids, maleic acids and fumaric acids and alkyl monoesters, comprising from 1 to 4 carbon atoms, of maleic or fumaric acids or anhydrides.

In one embodiment, the at least one basic comonomer may be aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer® and Lovocryl® 47 by the company National Starch, may also be used in the compositions disclosed herein.

(3) crosslinked and acylated polyamino amides partially or totally derived from polyamino amides of general formula: [symbol]
CO—R₁₀CO-Z [symbol] (II)
wherein:
R₁₀ is a divalent group derived from a saturated dicarboxylic acid, a mono- or dicarboxylic aliphatic acid comprising at least one ethylenic double bond, an ester of a lower alkanol, comprising from 1 to 6 carbon atoms, of these acids, and a group derived from the addition of at least one of the acids to amines chosen from bis(primary) or bis(secondary) amines; and
Z is chosen from bis(primary), mono- and bis(secondary) polyalkylene-polyamine groups and may, for example, be chosen from groups of the following formula:
   a) in proportions comprising from 60 to 100 mol %, the group
      —NH [symbol] (CH₂)ₓ—NH [symbol] ₚ (III)
   wherein x=2 and p=2 or 3, or alternatively x=3 and p=2
   this group being derived from a compound chosen from diethylenetriamine, triethlyenetetraamine and dipropylenetriamine;
   b) in proportions ranging from 0 to 40 mol %, the group (III) above wherein x=2 and p=1, wherein the groups is derived from a compound chosen from ethylenediamine and piperazine: 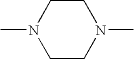
   c) in proportions comprising from 0 to 20 mol %, the —NH—(CH2)₆—NH— group derived from hexamethylenediamine, these polyamino amides being crosslinked by reaction addition of at least one difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids may be chosen from acids comprising from 6 to 10 carbon atoms, such as adipic acid, 2,2,4-trimethyladipic acid and 2,4,4-trimethyladipic acid, terephthalic acid, acids comprising at least one ethylenic double bond, such as acrylic acid, methacrylic acid and itaconic acid.

The alkane sultones used in the acylation may be chosen from propane sultone and butane sultone. The salts of the acylating agents may be chosen from sodium and potassium salts.

(4) polymers comprising at least one zwitterionic unit of formula: 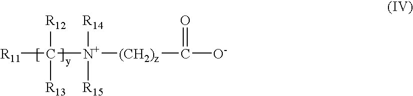
wherein:
R₁₁ is chosen from polymerizable unsaturated groups, such as acrylate, methacrylate, acrylamide and methacrylamide groups;
y and z, which may be identical or different, are each integers ranging from 1 to 3;
R₁₂ and R₁₃, which may be identical or different, are each chosen from a hydrogen atom, and methyl, ethyl and propyl groups; and
R₁₄ and R₁₅, which may be identical or different, are each chosen from a hydrogen atom and alkyl groups such that the sum of the carbon atoms in R₁₄ and R₁₅ does not exceed 10.

The polymers comprising at least one zwitterionic unit can also comprise at least one unit derived from non-zwitterionic monomers, such as monomer chosen from dimethyl and diethylaminoethyl acrylates and methacrylates, alkyl acrylates and methacrylates, acrylamides and methacrylamides, and vinyl acetate.

For example, the methyl methacrylate/methyl dimethylcarboxymethylammonioethylmethacrylate copolymers can be used in the compositions disclosed herein.

(5) chitosan-based polymers comprising monomer units corresponding to the formulae (D), (E) and (F) below: 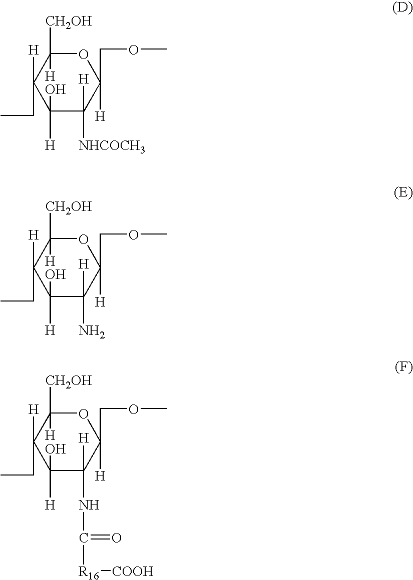
the unit (D) being present in proportions ranging from 0% to 30%, the unit (E) in proportions ranging from 5% to 50% and the unit (F) in proportions ranging from 30% to 90%, wherein in the unit (F), R₁₆ is a group of formula: 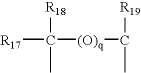
wherein:
if q=0, R₁₇, R₁₈ and R₁₉, which may be identical or different, are independently chosen from a hydrogen atom; methyl, hydroxyl, acetoxy and amino residues; monoalkylamine residues; and dialkylamine residues which are optionally interrupted by at least one nitrogen atom and optionally substituted with at least one group chosen from amine, hydroxyl, carboxyl, alkylthio and sulfonic groups; alkylthio residues wherein the alkyl group bears at least one amino residue, at least one of the groups R₁₇, R₁₈ and R₁₉ being, in this case, a hydrogen atom; or
if q=1, R₁₇, R₁₈ and R₁₉, which may be identical or different, are each chosen from a hydrogen atom, and, the salts formed by these compounds with bases or acids.

(6) polymers corresponding to the general formula (V) are described, for example, in French Patent No. FR 1 400 366: 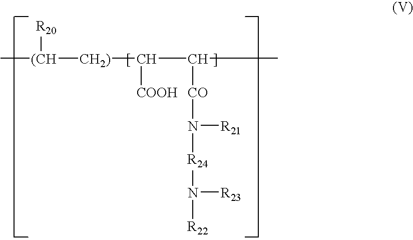
wherein:
R₂₀ is chosen from a hydrogen atom, a CH₃O group, a CH₃CH₂O group and a phenyl group;
R₂₁ is chosen from a hydrogen atom and lower alkyl groups, such as methyl and ethyl groups;
R₂₂ is chosen from a hydrogen atom and C₁-C₆ lower alkyl groups, such as methyl and ethyl groups; and
R₂₃ is chosen from C₁-C₆ lower alkyl groups, such as methyl and ethyl groups and groups corresponding to the formula: —R₂₄—N(R₂₂)₂, wherein R₂₄ is chosen from —CH₂—CH₂—, —CH₂—CH₂-CH₂— and —CH₂—CH(CH₃)— groups, R₂₂ is chosen from a hydrogen atom and C₁-C₆ lower alkyl groups, such as methyl and ethyl groups.

(7) polymers derived from the N-carboxyalkylation of chitosan, for example, N-carboxymethylchitosan and N-carboxybutylchitosan.

(8) amphoteric polymers of the type -D-X-D-X chosen from:
a) polymers obtained by the action of chloroacetic acid or sodium chloroacetate on compounds comprising at least one unit of formula:
   -D-X-D-X-D- (VI)
wherein
D is a group 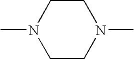
   and;
X is chosen from E and E′, wherein E and E′, which may be identical or different, are each chosen from divalent alkylene groups comprising at least one group chosen from straight and branched chains comprising up to 7 carbon atoms in the main chain, which are optionally substituted with at least one hydroxyl group and which can additionally comprise at least one oxygen, nitrogen or sulfur atom and 1 to 3 aromatic and/or heterocyclic rings; wherein the oxygen, nitrogen and sulfur atoms can be present in the form of at least one group chosen from ether, thioether, sulfoxide, sulfone, sulfonium, alkylamine and alkenylamine groups, hydroxyl, benzylamine, amine oxide, quaternary ammonium, amide, imide, alcohol, ester and urethane groups.
b) polymers of formula:
   -D-X-D-X- (VI′)
wherein:
D is a group 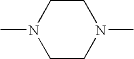
   and;
X is chosen from E and E′, wherein at least one X is E′; E having the meaning given above and E′ being chosen from divalent alkylene radicals comprising at least one chain chosen from straight and branched chains comprising up to 7 carbon atoms in the main chain, wherein the divalent alkylene radicals are optionally substituted with at least one hydroxyl group and comprise at least one nitrogen atom, the at least one nitrogen atom being substituted with an alkyl chain which is optionally interrupted by an oxygen atom, and wherein the alkyl chain comprises at least one functional group chosen from carboxyl functional groups and hydroxyl functional groups, and wherein the alkyl chain is betainized by reaction with a reactant chosen from chloroacetic acid and sodium chloroacetate.

(9) (C₁-C₅)alkyl vinyl ether/maleic anhydride copolymers partially modified by semiamidation with an N,N-dialkylaminoalkylamine such as N,N-dimethylamino-propylamine or by semiesterification with an N,N-dialkylaminoalkanol. These copolymers can also comprise other vinyl comonomers such as vinylcaprolactam.

Among the foregoing amphoteric fixing polymers, mention may be made of those of the family (3), such as the copolymers whose CTFA name is octylacryl-amide/acrylates/butylaminoethyl methacrylate copolymer, such as the products sold under the names Amphomer®, Amphomer® LV 71 or Lovocryl® 47 by the company National Starch and those of the family (4) such as methyl methacrylate/methyl dimethylcarboxy-methylammonioethylmethacrylate copolymers.

The nonionic polymers that may be used in the compositions disclosed herein are chosen from:
polyalkyloxazolines;
vinyl acetate homopolymers;
copolymers of vinyl acetate and of acrylic ester;
copolymers of vinyl acetate and of ethylene;
copolymers of vinyl acetate and of maleic ester, for example of dibutyl maleate;
copolymers of acrylic esters, for instance copolymers of alkyl acrylates and of alkyl methacrylates, such as the products sold by the company Rohm & Haas under the names Primale® AC-261 K and Eudragite® NE 30 D, by the company BASF under the name 8845, and by the company Hoechst under the name Appretan® N9212;
copolymers of acrylonitrile and of a nonionic monomer chosen, for example, from butadiene and alkyl (meth)acrylates; mention may be made of the products sold under the name CJ 0601 B by the company Rohm & Haas;
styrene homopolymers;
copolymers of styrene and of an alkyl (meth)acrylate, such as the products Mowilithe® LDM 6911, Mowilith® DM 611 and Mowilithe® LDM 6070 sold by the company Hoechst, and the products Rhodopas® SD 215 and Rhodopase® DS 910 sold by the company Rhône-Poulenc;
copolymers of styrene, of alkyl methacrylate and of alkyl acrylate;
copolymers of styrene and of butadiene;
copolymers of styrene, of butadiene and of vinylpyridine;
copolymers of alkyl acrylate and of urethane;
polyamides; and
vinyllactam homopolymers and copolymers.

The alkyl groups in the nonionic polymers mentioned may have from 1 to 6 carbon atoms.

The nonionic polymers disclosed herein may, for example, comprise vinyllactam units described in U.S. Pat. Nos. 3,770,683, 3,929,735, 4,521,504, 5,158,762 and 5,506,315 and in Patent Applications WO 94/121148, WO 96/06592 and WO 96/10593. They may be in pulverulent form or in the form of a solution or suspension.

The homopolymers or copolymers comprising vinyllactam units may, for example, be chosen from units of formula (IX): 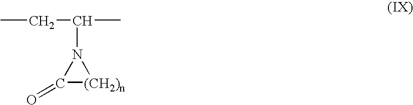
wherein n is independently 3, 4 or 5.

The number-average molar mass of the polymers comprising vinyllactam units may be, for example, greater than 5000, ranging from, by further example, 10 000 to 1 000 000 and ranging from, by even further example, 10 000 to 100 000.

Among homopolymers or copolymers comprising vinyllactam units, mention may be made of polyvinylpyrrolidones such as those sold under the name Luviskol® K30 by the company BASF; polyvinylcaprolactams such as those sold under the name Luviskol® PLUS by the company BASF; poly(vinylpyrrolidone/vinyl acetate) copolymers such as those sold under the name PVPVA® S630L by the company ISP, Luviskole® VA 73, VA 64, VA 55, VA 37 and VA 28 by the company BASF; and poly(vinylpyrrolidone/vinyl acetate/vinyl propionate) terpolymers, for instance those sold under the name Luviskol® VAP 343 by the company BASF.

The cationic polymers may also be chosen from polymers comprising at least one group chosen from primary, secondary, tertiary and quaternary amine groups forming part of the polymer chain or directly attached thereto, and may have a number-average molecular weight ranging from 500 to 5 000 000 such as from 1000 to 3 000 000.

The cationic polymers may also be chosen from:
(1) homopolymers or copolymers chosen from acrylic and methacrylic esters and amides and comprising at least one of the units of formula (VI), (VII), (Vil) or (IX) below: 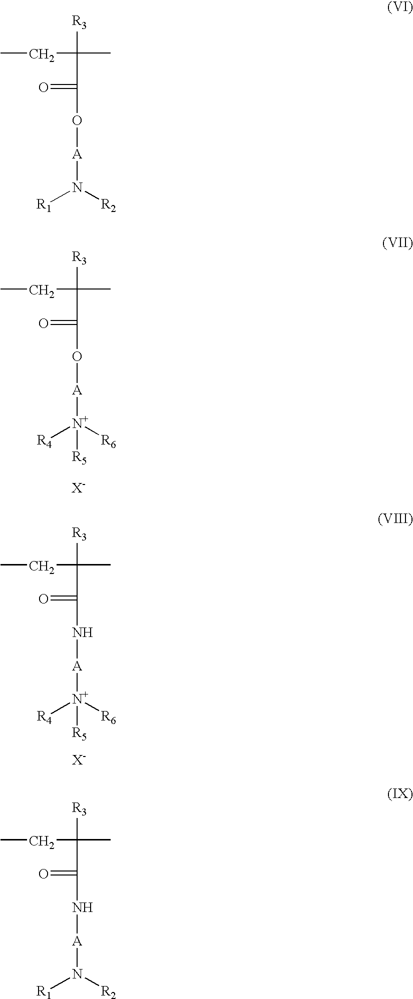
wherein:
R₁ and R₂, which may be identical or different, are each chosen from hydrogen and an alkyl group comprising from 1 to 6 carbon atoms, for example, methyl or ethyl;
R₃ is chosen from a hydrogen atom and a CH₃ radical;
R₄, R₅ and R₆, which may be identical or different, are chosen from an alkyl group ranging from 1 to 18 carbon atoms, such as an alkyl group having from 1 to 6 carbon atoms, and a benzyl radical;
A is chosen from linear and branched alkyl groups having from 1 to 6 carbon atoms, such as 2 or 3 carbon atoms and hydroxyalkyl groups having from 1 to 4 carbon atoms; and
X⁻ is chosen from an anion derived from inorganic and organic acids, such as a methosulfate anion or a halide such as chloride or bromide.

The polymers of family (1) may also comprise at least one unit derived from comonomers chosen from acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinyl-caprolactam, and vinyl esters.

The polymers of family (1), may be, for example:
copolymers of acrylamide and of dimethylaminoethyl methacrylate quaternized with dimethyl sulfate or with a dimethyl halide, such as the product sold under the name Hercofloc by the company Hercules,
the copolymers of acrylamide and of methacryloyloxyethyltrimethyl-ammonium chloride described, for example, in Patent Application EP-A-080 976 and sold under the name Bina Quat P 100 by the company Ciba Geigy,
the copolymer of acrylamide and of methacryloyloxyethyltrimethylammonium methosulfate sold under the name Reten by the company Hercules,
quaternized or non-quaternized vinylpyrrolidone/dialkylaminoalkyl acrylate or methacrylate copolymers, such as the products sold under the name “Gafquat” by the company ISP, such as, for example, “Gafquat 734” or “Gafquat 755”, or alternatively the products known as “Copolymer 845, 958 and 937”. These polymers are described in French Patent Nos. 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the product sold under the name Styleze CC 10 by ISP, and
quaternized vinylpyrrolidone/dimethylaminopropyl methacrylamide copolymers such as the product sold under the name “Gafquat HS 100” by the company ISP.

(2) cellulose ether derivatives comprising quaternary ammonium groups, described in French Patent No. 1 492 597, and polymers sold under the name “JR” (JR 400, JR 125 and JR 30M) and “LR” (LR 400 or LR 30M) by the company Union Carbide Corporation. These polymers are also defined in the CTFA dictionary as quaternary ammoniums of hydroxyethylcellulose that have reacted with an epoxide substituted with a trimethylammonium group.

(3) cationic cellulose derivatives such as cellulose copolymers or cellulose derivatives grafted with a water-soluble quaternary ammonium monomer, and described in U.S. Pat. No. 4,131,576, such as hydroxyalkylcelluloses, for instance hydroxymethyl-, hydroxyethyl- or hydroxypropylcelluloses grafted, including those with a methacryloylethyltrimethylammonium, methacrylamidopropyltrimethylammonium or dimethyldiallylammonium salt. The commercial products corresponding to this definition are the products sold under the names “Celquat L 200” and “Celquat H 100” by the company National Starch.

(4) cationic polysaccharides described in U.S. Pat. Nos. 3,589,578 and 4 031 307, such as guar gums containing cationic trialkylammonium groups. Guar gums modified with a salt (e.g. chloride) of 2,3-epoxypropyltrimethylammonium are used, for example. Such products are sold under the trade names Jaguar C13 S, Jaguar C 15, Jaguar C 17 and Jaguar C162 by the company Meyhall.

(5) polymers chosen, for example, from piperazinyl units and divalent alkylene or hydroxyalkylene radicals comprising straight and branched chains, optionally interrupted by oxygen, sulfur or nitrogen atoms and by aromatic or heterocyclic rings, as well as the oxidation and quaternization products of these polymers. Such polymers are described in French Patent Nos. 2 162 025 and 2 280 361.

(6) water-soluble polyamino amides prepared by polycondensation of an acidic compound with a polyamine; optionally, the polyamino amides may be crosslinked compounds chosen from epihalohydrins, diepoxides, dianhydrides, unsaturated dianhydrides, bis-unsaturated derivatives, bis-halohydrins, bis-azetidiniums, bis-haloacyidiamines, bis-alkyl halides and optionally with an oligomer resulting from the reaction of a difunctional compound which is reactive with compounds chosen from bis-halohydrins, bis-azetidiniums, bis-haloacyldiamines, bis-alkyl halides, epihalohydrins, diepoxides and bis-unsaturated derivatives; the crosslinking agent being used in proportions ranging from 0.025 to 0.35 mol per amine group of the polyamino amide; these polyamino amides may be alkylated or, if they contain at least one tertiary amine functions, they may be quaternized. Such polymers are described in French Patent Nos. 2 252 840 and 2 368 508.

(7) polyamino amide derivatives resulting from the condensation of polyalkylene polyamines with polycarboxylic acids followed by alkylation with difunctional agents. Adipic acid/dialkylaminohydroxyalkyldialkylenetriamine polymers in which the alkyl radical comprises from 1 to 4 carbon atoms and, for example, denotes methyl, ethyl or propyl are described in French Patent No. 1 583 363. These derivatives may be, for example, the adipic acid/dimethylamino-hydroxypropyl/diethylenetriamine polymers sold under the name Cartaretine F, F4 and F8 by the company Sandoz.

(8) polymers obtained by reaction of a polyalkylene polyamine comprising two primary amine groups and at least one secondary amine group with a dicarboxylic acid chosen from diglycolic acids and saturated aliphatic dicarboxylic acids comprising from 3 to 8 carbon atoms. The molar ratio between the polyalkylene polyamine and the dicarboxylic acid may range from 0.8:1 to 1.4:1; the polyamino amide resulting therefrom is reacted with epichlorohydrin in a molar ratio of epichlorohydrin relative to the secondary amine group of the polyamino amide ranging from 0.5:1 to 1.8:1. Such polymers are described in U.S. Pat. Nos. 3,227,615 and 2,961,347. Polymers of this type are sold under the name Hercosett 57 by the company Hercules Inc. and under the name PD 170 or Delsette 101 by the company Hercules in the case of the adipic acid/epoxypropyl/diethylenetriamine copolymer.

(9) quaternary polymers of vinylpyrrolidone and of vinylimidazole, such as, for example, the products sold under the names Luviquat FC 905, FC 550 and FC 370 by the company BASF.

Other cationic polymers that can be used in the compositions disclosed herein may be chosen from polyalkyleneimines, for example, polyethyleneimines; polymers containing vinylpyridine or vinylpyridinium units; condensates of polyamines and of epichlorohydrin; quaternary polyureylene; and chitin derivatives.

Hydrocarbon-based polymers containing silicone grafts and silicones containing hydrocarbon-based grafts in the compositions disclosed herein. It is also possible to use polyurethanes other than the cationic polyurethanes of elastic nature. These different compounds may be nonionic, cationic, anionic or amphoteric.

A polyurethane used in the compositions disclosed herein may also comprise at least one polysiloxane block and its base repeating unit corresponds, for example, to the general formula (VII):
—O—P—O—CO—NH—R—NH—CO— (VII)
wherein:
P is a polysiloxane segment; and
R is a divalent group chosen from alkylene groups of aromatic, substituted and unsubstituted C₁ to C₂₀ aliphatic and substituted and unsubstituted C₁ to C₂₀ cycloaliphatic type.

The polysiloxane segment P may also be chosen, for example, from units of the formula (VIII): 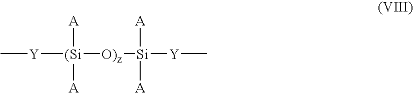
wherein:
A, which may be identical or different, is chosen from C₁-C₂₀ monovalent hydrocarbon-based groups free or substantially free of ethylenic unsaturation and aromatic groups,
Y is a divalent hydrocarbon-based group, and
z is an integer chosen such that the average molecular mass of the polysiloxane segment ranges from 300 to 10 000.

The divalent group Y may be chosen from the alkylene groups of formula —(CH₂)ₐ—, wherein a is an integer ranging from 1 to 10.

A may be chosen from alkyl groups, for example methyl, ethyl, propyl, isopropyl, butyl, pentyl, hexyl, octyl, decyl, dodecyl and octadecyl groups; cycloalkyl groups, for example, cyclohexyl group; aryl groups, for example, phenyl and naphthyl; and arylalkyl groups, for example, benzyl, phenylethyl, tolyl and xylyl groups.

Examples of fixing polyurethanes in the composition disclosed herein include the dimethylolpropionic acid/isophorone diisocyanate/neopentyl glycol/polyesterdiols copolymer (also known under the name polyurethane-1, INCI name) sold under the brand name Luviset® PUR by the company BASF, and the dimethylolpropionic acid/isophorone diisocyanate/neopentyl glycol/polyesterdiols/silicone diamine copolymer (also known under the name polyurethane-6, INCI name) sold under the brand name Luviset® Si PUR A by the company BASF.

The concentration of additional fixing polymer in the composition disclosed herein ranges, for example, from 0.05% to 10%, such as from 0.1% to 5% and even further, from 0.2% to 3% by weight relative to the total weight of the composition.

The compositions disclosed herein may optionally contain at least one thickening polymer, also known as a “rheology modifier”.

The at least one rheology modifier may be chosen from fatty acid amides (for example, coconut diethanolamide or monoethanolamide, or oxyethylenated monoethanolamide of carboxylic acid alkyl ether), cellulose-based thickeners (for example, hydroxyethylcellulose, hydroxypropylcellulose or carboxymethylcellulose), guar gum and its derivatives (for example, hydroxypropyl guar), gums of microbial origin (for example, xanthan gum, scleroglucan gum), crosslinked homopolymers of acrylic acid or of acrylamidopropanesulfonic acid, and associative thickening polymers as described below.

The associative polymers may be water-soluble polymers capable, in an aqueous medium, of reversibly combining with each other or with other molecules.

Their chemical structure comprises hydrophilic zones and hydrophobic zones characterized by at least one fatty chain.

The associative polymers may be chosen from anionic, cationic, amphoteric and nonionic polymers.

Their concentration may range, for example, from 0.01% to 10% and further, for example, from 0.1% to 5% by weight relative to the total weight of the composition disclosed herein.

The associative anionic polymers may be chosen from:
(I) polymers comprising at least one hydrophilic unit and at least one fatty-chain allyl ether unit, for example, polymers whose hydrophilic unit comprises an ethylenic unsaturated anionic monomer, for example, monomers chosen from vinylcarboxylic acids, acrylic acids, methacrylic acids and mixtures thereof, the fatty-chain allyl ether unit corresponding to the monomer of formula (XV) below:
   CH₂═CR′CH₂OBₙR (XV)
wherein:
   R′ is chosen from H or CH₃;
   B is an ethyleneoxy radical;
   n is an integer ranging from 1 to 100;
   R is a hydrocarbon-based radical chosen from alkyl, arylalkyl, aryl, alkylaryl and cycloalkyl radicals, comprising from 8 to 30 carbon atoms, such as from 10 to 24 carbon atoms and even further from 12 to 18 carbon atoms.

A unit of formula (XV), for example, is a unit in which R′ denotes H, n is equal to 10 and R denotes a stearyl (C₁₈) radical.

The anionic associative polymers disclosed herein may be described and prepared, according to an emulsion polymerization process, in Patent EP-0 216 479.

The anionic associative polymers disclosed herein may also be polymers comprising acrylic acids and/or of methacrylic acids ranging from 20% to 60% by weight, lower alkyl (meth)acrylates ranging from 5% to 60% by weight, fatty-chain allyl ethers of formula (XV) ranging from 2% to 50% by weight, and crosslinking agents ranging from 0% to 1% by weight, wherein the crosslinking agent is chosen from copolymerizable unsaturated polyethylenic monomers, for instance diallyl phthalate, allyl (meth)acrylate, divinylbenzene, (poly)ethylene glycol dimethacrylate or methylenebisacrylamide.

The anionic associative polymers, for example, may be chosen from crosslinked terpolymers of methacrylic acids, ethyl acrylates and polyethylene glycol (10 EO) stearyl alcohol ethers (Steareth-10), for example, those sold by the company Allied Colloids under the names Salcare SC 80® and Salcare SC 90®, which are aqueous 30% emulsions of a crosslinked terpolymer of methacrylic acid, of ethyl acrylate and of steareth-10 allyl ether (40/50/10).
(II) polymers comprising at least one hydrophilic unit of unsaturated olefinic carboxylic acid type, and at least one hydrophobic unit of (C₁₀-C₃₀)alkyl ester of unsaturated carboxylic acid type.

In one embodiment, these polymers are chosen from the monomer of formula (XVI): 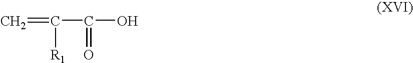
wherein:
   R₁ is chosen from H, CH₃, and C₂H₅; and
the hydrophobic unit of (C₁₀-C₃₀)alkyl ester of unsaturated carboxylic acid type corresponds to the monomer of formula (XVII): 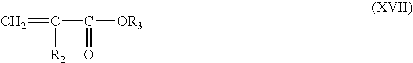
wherein:
   R₂ is chosen from H (e.g., acrylate units), CH₃, (e.g., methacrylate units), and C₂H₅ (e.g., ethacrylate units), and
   R₃ is chosen from a C₁₀-C₃₀ alkyl, for example, a C₁₂-C₂₂ alkyl radical.

(C₁₀-C₃₀) alkyl esters of unsaturated carboxylic acids may be chosen from, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers as disclosed herein are described and prepared, for example, according to U.S. Pat. Nos. 3,915,921 and 4,509,949.

Anionic associative polymers, for example are polymers formed from a monomer mixture comprising:
(i) acrylic acid,
(ii) an ester of formula (XVII) described above in which R₂ is chosen from H and CH₃, R₃ is chosen from an alkyl radical comprising from 12 to 22 carbon atoms, and
(iii) a crosslinking agent, chosen from a copolymerizable unsaturated polyethylenic monomer, for instance diallyl phthalate, allyl (meth)acrylate, divinylbenzene, (poly)ethylene glycol dimethacrylate and methylenebisacrylamide.

Anionic associative polymers used in the compositions disclosed herein may comprise from 95% to 60% by weight of acrylic acid (hydrophilic unit), 4% to 40% by weight of C₁₀-C₃₀ alkyl acrylate (hydrophobic unit) and 0% to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Anionic associative polymers may be chosen from, for example, the products sold by the company Goodrich under the trade names Pemulen TR1®, Pemulen TR2® and Carbopol 1382®, and Pemulen TR1®, and the product sold by the company SEPPIC under the name Coatex SX®.
(III) maleic anhydride/C30-C₃₈ α-olefin/alkyl maleate terpolymers, such as the product (maleic anhydride/C₃₀-C₃₈ α-olefin/isopropyl maleate copolymer) sold under the name Performa V 1608® by the company Newphase Technologies.
(IV) acrylic terpolymers comprising:
(a) a carboxylic acid comprising α,β-monoethylenic unsaturation in an amount ranging from 20% to 70% by weight,
(b) a non-surfactant monomer comprising α,β-monoethylenic unsaturation other than (a) in an amount ranging from 20% to 80% by weight,
(c) a nonionic monourethane which is the product of reaction of a monohydric surfactant with a monoisocyanate containing monoethylenic unsaturation in an amount ranging from 0.5% to 60% by weight,

Non-limiting examples of such terpolymers include those described in Patent Application EP-A-0 173 109 and the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
(V) copolymers comprising among their monomers a carboxylic acid containing α,β-monoethylenic unsaturation and an ester of a carboxylic acid containing α,β-monoethylenic unsaturation and of an oxyalkylenated fatty alcohol.

These compounds may also comprise as a monomer an ester of a carboxylic acid containing α,β-monoethylenic unsaturation and of a C₁-C₄ alcohol.

An example of a compound of this type is Aculyn 22® sold by the company Rohm & Haas, which is a methacrylic acid/ethyl acrylate/stearyl methacrylate oxyalkylenated terpolymer.

The associative polymers of cationic type may, for example, be chosen from:
(I) cationic associative polyurethanes, described by the present inventors in French Patent Application No. 00/09609, may be represented by formula (XVIII):
   R—X—(P)ₙ—[L-(Y)m]r-L′-(P′)ₚ—X′—R′ (XVIII)
wherein:
R and R′, which may be identical or different, are each chosen from a hydrophobic group or a hydrogen atom;
X and X′, which may be identical or different, are each chosen from groups comprising an amine function optionally bearing a hydrophobic group, or alternatively a group L″;
L, L′ and L″, which may be identical or different, are each chosen from groups derived from a diisocyanate;
P and P′, which may be identical or different, are each chosen from groups comprising an amine function optionally bearing a hydrophobic group;
Y is a hydrophilic group;
r is an integer ranging from 1 to 100, such as from 1 to 50 and further, for example, ranging from 1 to 25;
n, m and p are each chosen from a number ranging from 0 to 1000;
the molecule comprising at least one protonated or quaternized amine function and at least one hydrophobic group.

In one embodiment of these polyurethanes, the only hydrophobic groups may be the groups R and R′ at the chain ends.

For example, one family of cationic associative polyurethanes may be the one corresponding to formula (XVIII) wherein:

R and R′, which may be identical or different, are each chosen from a hydrophobic group;
X and X′ are each chosen from a group L″;
n and p are each chosen from a number ranging from 1 to 1000; and
L, L′, L″, P, P′, Y and m are defined as above.

Another family of cationic associative polyurethanes, for example, is the one corresponding to formula (XVIII) wherein:
R and R′, which may be identical or different, are each chosen from a hydrophobic group;
X and X′ are each chosen from a group L″;
n and p are 0; and
L, L′, L″, Y and m are defined as above.

The fact that n and p are 0 means that these polymers do not comprise units derived from a monomer containing an amine function incorporated into the polymer during the polycondensation. The protonated amine functions of these polyurethanes result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide, a sulfate, etc.

Another family of cationic associative polyurethanes is the one corresponding to formula (Ia) wherein:

R and R′, which may be identical or different, are each chosen from a hydrophobic group;
X and X′, which may be identical or different, are each chosen from a group comprising a quaternary amine;
n and p are zero; and
L, L′, Y and m are defined as above.

The number-average molecular mass of the cationic associative polyurethanes, may range from 400 to 500 000, such as from 1000 to 400 000 and even from 1000 to 300 000.

As defined herein, the expression “hydrophobic group” is a radical or polymer chosen from saturated and unsaturated, linear and branched hydrocarbon-based chains, which may contain at least one hetero atoms such as P, O, N or S, or a radical containing a perfluoro or silicone chain. When the hydrophobic group is a hydrocarbon-based radical, it comprises at least 10 carbon atoms, for example, from 10 to 30 carbon atoms, further, for example, from 12 to 30 carbon atoms and even further, for example, from 18 to 30 carbon atoms.

For example, the hydrocarbon-based group may be derived from a monofunctional compound.

The hydrophobic group may also be derived from a fatty alcohol such as stearyl alcohol, dodecyl alcohol or decyl alcohol. It may also be a hydrocarbon-based polymer such as polybutadiene.

When X and X′ are groups comprising a tertiary or quaternary amine, X and X′ is chosen from the following: 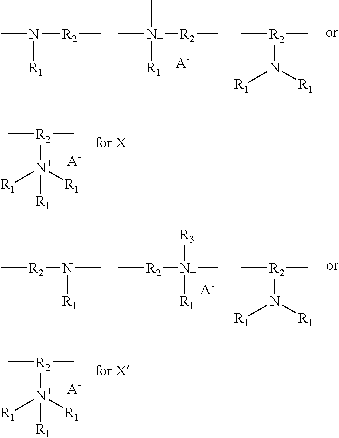
wherein:
R₂ is chosen from linear and branched alkylene radicals comprising from 1 to 20 carbon atoms, optionally chosen from saturated and unsaturated rings, and arylene radicals, wherein at least one of the carbon atoms is optionally replaced with a hetero atom chosen from N, S, O and P;
R₁ and R₃, which may be identical or different, are chosen from linear and branched C₁-C₃₀ alkyl and alkenyl radicals and aryl radicals, wherein at least one of the carbon atoms is optionally replaced with a hetero atom chosen from N, S, O and P;
A⁻ is a physiologically acceptable counterion.

L, L′ and L″ are chosen from: 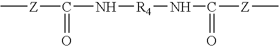
wherein:
Z is chosen from —O—, —S— and —NH—; and
R₄ is chosen from linear and branched alkylene radicals having from 1 to 20 carbon atoms, optionally chosen from saturated and unsaturated rings, and arylene radicals, wherein at least one of the carbon atoms is optionally replaced with a hetero atom chosen from N, S, O and P.

P and P′ comprising an amine function are chosen from: 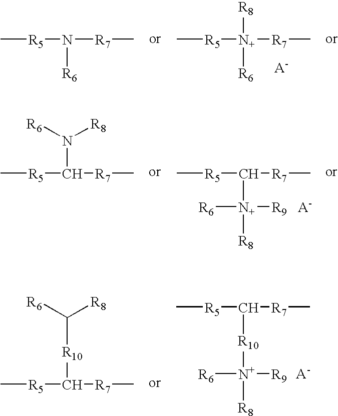
wherein:
R₅ and R₇ have the same meanings as R₂ defined above;
R₆, R₈ and R₉ have the same meanings as R₁ and R₃ defined above;
R₁₀ is chosen from linear and branched, optionally unsaturated alkylene group optionally comprising at least one hetero atoms chosen from N, O, S and P; and
A⁻ is a physiologically acceptable counterion.

In Y, the term “hydrophilic group” is a polymeric or non-polymeric water-soluble group.

For example, when Y is not a polymer, Y may be chosen from ethylene glycol, diethylene glycol and propylene glycol.

When Y is a hydrophilic polymer, Y may be chosen from polyethers, sulfonated polyesters, sulfonated polyamides or a mixture of these polymers. For example, the hydrophilic compound may be a polyether, a poly(ethylene oxide) or poly(propylene oxide).

The cationic associative polyurethanes of formula (XVIII) may be formed from groups chosen from diisocyanates and from various compounds with functions containing a labile hydrogen. The functions containing a labile hydrogen may be chosen from alcohol, primary or secondary amine or thiol functions, giving, after reaction with the diisocyanate functions, polyurethanes, polyureas and polythioureas, respectively. The expression “polyurethanes disclosed herein” encompasses these three types of polymers, namely polyurethanes per se, polyureas and polythioureas, and also copolymers thereof.

A first type of compound involved in the preparation of the polyurethane of formula (XVIII) is a compound comprising at least one unit containing an amine function. This compound may be multifunctional, for example, difunctional, wherein the compound comprises two labile hydrogen atoms chosen from, for example, a hydroxyl, primary amine, secondary amine and thiol function. A mixture of multifunctional and difunctional compounds wherein the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae:
HZ-(P)ₙ-ZH
and
HZ-(P′)ₚ-ZH
wherein:
Z, P, P′, n and p are as defined above.

For example, compounds containing an amine function may be chosen from N-methyldiethanolamine, N-tert-butyidiethanolamine and N-sulfoethyldiethanolamine.

The second compound involved in the preparation of the polyurethane of formula (XVIII) is a diisocyanate corresponding to the formula:
O═C═N—R₄—N═C═O
in which R₄ is as defined above.

For example, the diisocyanate may be chosen from methylenediphenyl diisocyanate, methylenecyclohexane diisocyanate, isophorone diisocyanate, toluene diisocyanate, naphthalene diisocyanate, butane diisocyanate and hexane diisocyanate.

A third compound involved in the preparation of the polyurethane of formula (XVIII) is a hydrophobic compound intended to form the terminal hydrophobic groups of the polymer of formula (XVIII).

This hydrophobic compound consists of a hydrophobic group and of a function containing a labile hydrogen chosen from, for example, a hydroxyl, primary or secondary amine, and thiol function.

For example, this hydrophobic compound may be a fatty alcohol such as, stearyl alcohol, dodecyl alcohol or decyl alcohol. When this compound comprises a polymeric chain, it may be, for example, α-hydroxylated hydrogenated polybutadiene.

The hydrophobic group of the polyurethane of formula (XVIII) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q is a leaving group, for example, a halide, or a sulfate.

The cationic associative polyurethane may also comprise a hydrophilic block. This hydrophilic block is provided by a fourth type of compound involved in the preparation of the polymer. This compound may be multifunctional, for example, difunctional. A mixture of multifunctional and difunctional compounds wherein the percentage of multifunctional compounds is low may also be used.

The functions containing a labile hydrogen are chosen from an alcohol, a primary or secondary amine or a thiol functions. This fourth compound may be a polymer terminated at the chain ends with one of these functions containing a labile hydrogen.

For example, when the fourth compound is not a polymer, it may be chosen from ethylene glycol, diethylene glycol and propylene glycol.

When the fourth compound is a hydrophilic polymer it may be chosen from, for example, polyethers, sulfonated polyesters, sulfonated polyamides, and mixtures thereof. The hydrophilic compound may be, for example, poly(ethylene oxide) or poly(propylene oxide).

The hydrophilic group termed Y in formula (XVIII) is optional. Specifically, the units containing a quaternary amine or protonated function may suffice to provide the solubility or water-dispersibility required for this type of polymer in an aqueous solution.
(II) quaternized cellulose derivatives and polyacrylates containing non-cyclic amine side groups.

The quaternized cellulose derivatives may be chosen from:
quaternized celluloses modified with groups comprising at least one fatty chain, such as alkyl, arylalkyl or alkylaryl groups comprising at least 8 carbon atoms, or mixtures thereof; and
quaternized hydroxyethylcelluloses modified with groups comprising at least one fatty chain, such as alkyl, arylalkyl or alkylaryl groups comprising at least 8 carbon atoms, or mixtures thereof.

The alkyl radicals borne by the above quaternized celluloses or hydroxyethylcelluloses may contain from 8 to 30 carbon atoms. The aryl radicals may, for example, be chosen from phenyl, benzyl, naphthyl and anthryl groups.

Examples of quaternized alkylhydroxyethylcelluloses containing C₈-C₃₀ fatty chains may be chosen from, for example, the products Quatrisoft LM 200®, Quatrisoft LM-X 529-18-A®, Quatrisoft LM-X 529-18B® (C₁₋₂ alkyl) and Quatrisoft LM-X 529-8′ (C₁₋₈ alkyl) sold by the company Amerchol, and the products Crodacel QM®, Crodacel QL® (C₁₋₂ alkyl) and Crodacel QS® (C₁₋₈ alkyl) sold by the company Croda.

The amphoteric associative polymers may be chosen from those comprising at least one non-cyclic cationic unit, for example, polymers prepared from or comprising a fatty-chain monomer ranging, for example, from 1 to 20 mol %, such as from 1.5 to 15 mol % and even further from 1.5 to 6 mol % relative to the total number of moles of monomers.

The amphoteric associative polymers may also comprise or may be prepared by copolymerizing:
1) at least one monomer of formula (XIX) or (XX): 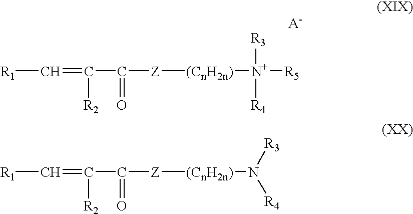
wherein:
   R₁ and R₂, which may be identical or different, are each chosen from a hydrogen atom and a methyl radical;
   R₃, R₄ and R₅, which may be identical or different, are each chosen from linear and branched alkyl radical comprising from 1 to 30 carbon atoms;
      Z is chosen from NH groups and an oxygen atoms;
      n is an integer ranging from 2 to 5; and
      A⁻ is an anion derived from an organic or mineral acid, such as a methosulfate anion or a halide such as chloride or bromide;
2) at least one monomer of formula (XXI)
   R₆—CH═CR₇—COOH (XXI)
wherein R₆ and R₇, which may be identical or different, are chosen from a hydrogen atom and a methyl radical; and
3) at least one monomer of formula (XXII):
   R₆—CH═CR₇—COXR₈ (XXII)
wherein:
   R₆ and R₇, which may be identical or different, are chosen from a hydrogen atom and a methyl radical;
   X is chosen from an oxygen or nitrogen atom; and
   R₈ is chosen from linear and branched alkyl radicals comprising from 1 to 30 carbon atoms;
wherein at least one of the monomers of formula (XIX), (XX) or (XXII) comprises at least one fatty chain.

The monomers of formulae (XIX) and (XX) may be chosen, for example, from:
dimethylaminoethyl methacrylate, dimethylaminoethyl acrylate,
diethylaminoethyl methacrylate, diethylaminoethyl acrylate,
dimethylaminopropyl methacrylate, dimethylaminopropyl acrylate, and
dimethylaminopropylmethacrylamide, dimethylaminopropylacrylamide, these monomers optionally being quaternized, for example with a C₁-C₄ alkyl halide or a C₁-C₄ dialkyl sulfate.

By further example, the monomer of formula (XIX) may be chosen from acrylamidopropyltrimethylammonium chloride and methacrylamidopropyl-trimethylammonium chloride.

The monomers of formula (XXI) may chosen from acrylic acid, methacrylic acid, crotonic acid and 2-methylcrotonic acid.

The monomers of formula (XXII) may be chosen from C₁₂-C₂₂, for example, C₁₆-C₁₈ alkyl acrylates and methacrylates.

The monomers constituting the fatty-chain amphoteric polymers may also be already neutralized and/or quaternized.

The ratio of the number of cationic charges/anionic charges may be, for example, equal to 1.

The amphoteric associative polymers may comprise a fatty chain (monomer of formula (XIX), (XX) or (XXII) ranging from 1 mol % to 10 mol % of the monomer, for example, 1.5 mol % to 6 mol %.

The weight-average molecular weights of the amphoteric associative polymers disclosed herein may range from 500 to 50 000 000 for example, from 10 000 to 5 000 000.

The amphoteric associative polymers disclosed herein may also contain other monomers such as nonionic monomers, for example, C₁-C₄ alkyl acrylates or methacrylates.

Amphoteric associative polymers disclosed herein are described and prepared, for example, in Patent Application WO 98/44012.

The amphoteric associative polymers disclosed herein, may be chosen from, for example, acrylic acid/(meth)acrylamidopropyltrimethylammonium chloride/stearyl methacrylate terpolymers.

The associative polymers of nonionic type may be chosen from:
(1) celluloses modified with groups comprising at least one fatty chain chosen from, for example:
   hydroxyethylcelluloses modified with groups comprising at least one fatty chain, such as alkyl, arylalkyl or alkylaryl groups, or mixtures thereof, and wherein the alkyl groups are, for example, C₈-C₂₂, by further example, the product Natrosol Plus Grade 330 CS® (C₁₋₆ alkyls) sold by the company Aqualon, or the product Bermocoll EHM 100® sold by the company Berol Nobel,
   those modified with alkylphenyl polyalkylene glycol ether groups, such as the product Amercell Polymer HM-1500® (nonylphenyl polyethylene glycol (15) ether) sold by the company Amerchol.
(2) hydroxypropyl guars modified with groups comprising at least one fatty chain, such as the product Esaflor HM 22® (C₂₋₂ alkyl chain) sold by the company Lamberti, and the products RE210-18®(C₁₋₄ alkyl chain) and RE205-1® (C₂₋₀ alkyl chain) sold by the company Rhone-Poulenc.
(3) copolymers of vinylpyrrolidone and of fatty-chain hydrophobic monomers chosen from, for example:
   the products Antaron V216® or Ganex V216® (vinylpyrrolidone/hexadecene copolymer) sold by the company I.S.P.
   the products Antaron V220® or Ganex V220® (vinylpyrrolidone/eicosene copolymer) sold by the company I.S.P.
(4) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208®.
(5) copolymers of hydrophilic methacrylates or acrylates and of hydrophobic monomers comprising at least one fatty chain, such as, for example, the polyethylene glycol methacrylate/lauryl methacrylate copolymer.
(6) polyurethane polyethers comprising in their chain both hydrophilic blocks usually of polyoxyethylenated nature and hydrophobic blocks which may be aliphatic sequences alone and/or cycloaliphatic and/or aromatic sequences.
(7) polymers with an aminoplast ether skeleton comprising at least one fatty chain, such as the Pure Thix® compounds sold by the company Sud-Chemie.

The polyurethane polyethers may comprise at least two hydrocarbon-based lipophilic chains comprising from 6 to 30 carbon atoms, separated by a hydrophilic block, the hydrocarbon-based chains optionally having at least one pendent chains, or chains at the end of the hydrophilic block. In addition, the polymer may comprise a hydrocarbon-based chain at one end or at both ends of a hydrophilic block.

The polyurethane polyethers may be multiblock, for example, triblock form. Hydrophobic blocks may, for example, be at each end of the chain (for example: triblock copolymer with a hydrophilic central block) or distributed both at the ends and in the chain (for example: multiblock copolymer). These same polymers may also be chosen, for example, from graft polymers and starburst polymers.

The nonionic fatty-chain polyurethane polyethers may be, for example, triblock copolymers in which the hydrophilic block is a polyoxyethylenated chain comprising from 50 to 1 000 oxyethylene groups. The nonionic polyurethane polyethers comprise a urethane linkage between the hydrophilic blocks, whence arises the name.

The nonionic fatty-chain may also be chosen from polyurethane polyethers wherein the hydrophilic blocks are linked to the lipophilic blocks via other chemical bonds.

Nonionic fatty-chain polyurethane polyethers may be chosen from, for example, Rheolate 205® containing a urea function, sold by the company Rheox, Rheolate® 208, 204 or 212, Acrysol RM 184®, Elfacos T210 ® containing a C₁₂₋₁₄ alkyl chain, the product Elfacos T212® containing a C₁₈ alkyl chain, from Akzo, DW 1206BE from Rohm & Haas containing a C₂₀ alkyl chain and a urethane linkage, sold at a solids content of 20% in water may be used. In addition, solutions or dispersions of these polymers, in water or in aqueous-alcoholic medium, for example, Rheolate® 255, Rheolatee 278 and Rheolatee 244 sold by the company Rheox. and DW 1206F and DW 1206J sold by the company Rohm & Haas, may also be used.

The polyurethane polyethers that may be used as disclosed herein are described in the article by G. Formum, J. Bakke and Fk. Hansen—Colloid Polym. Sci 271, 380.389 (1993).

The polyurethane polyethers that may be used as disclosed herein may be obtained by polycondensation of at least three compounds chosen from (i) at least one polyethylene glycol ranging from 150 to 180 mol of ethylene oxide, (ii) stearyl alcohol and decyl alcohol, and (iii) at least one diisocyanate.

Such polyurethane polyethers are sold by the company Rohm & Haas under the names Aculyn 44® and Aculyn 46® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl-isocyanate) (SMDI), at 35% by weight in a mixture of propylene glycol (39%) and water (26%)].

The composition disclosed herein may also contain at least one surfactant.

The at least one surfactant may be chosen from, for example:

(i) Anionic Surfactant(s):

Anionic surfactants that may be used, alone or as mixtures, may be chosen from:
salts, for example alkali metal salts chosen from, for example, sodium salts, ammonium salts, amine salts, amino alcohol salts and magnesium salts, of the compounds chosen from, for example: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; (C₆-C₂₄)alkyl sulfosuccinates, (C₆-C₂₄)alkyl ether sulfosuccinates, (C₆-C₂₄)alkylamide sulfosuccinates; (C₆-C₂₄)alkyl sulfoacetates; (C₆-C₂₄)acyl sarcosinates; and (C₆-C₂₄)acyl glutamates;
(C₆-C₂₄)alkylpolyglycoside carboxylic esters chosen from alkylglucoside citrates, alkylpolyglycoside tartrates and alkylpolyglycoside sulfosuccinates, alkylsulfo-succinamates; acyl isethionates and N-acyl taurates; the alkyl or acyl radical of all of these different compounds comprising from 12 to 20 carbon atoms; and the aryl radical, for example, a phenyl or benzyl group;
fatty acid salts chosen from, for example, oleic, ricinoleic, palmitic and stearic acid salts; coconut oil acid or hydrogenated coconut oil acid; and acyl lactylates wherein the acyl radical contains 8 to 20 carbon atoms;
alkyl D-galactoside uronic acids and their salts; and
polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, for example, those comprising from 2 to 50 alkylene oxide groups, by further example, ethylene oxide groups, and mixtures thereof.

(ii) Nonionic Surfactant(s):

The nonionic surfactants, for example described in “Handbook of Surfactants” by M. R. Porter, published by Blackie & Son (Glasgow and London), 1991, pp. 116-178), may be chosen from polyethoxylated or polypropoxylated alkylphenols; alpha-diols or alcohols, having a fatty chain comprising, for example, 8 to 18 carbon atoms, by further example, ethylene oxide or propylene oxide groups ranging from 2 to 50. Mention may also be made of copolymers of ethylene oxide and of propylene oxide; condensates of ethylene oxide and of propylene oxide with fatty alcohols; polyethoxylated fatty amides ranging from 2 to 30 mol of ethylene oxide; polyglycerolated fatty amides ranging from 1 to 5, for example, from 1.5 to 4, glycerol groups; oxyethylenated fatty acid esters of sorbitan ranging from 2 to 30 mol of ethylene oxide; fatty acid esters of sucrose; fatty acid esters of polyethylene glycol; alkylpolyglycosides; N-alkylglucamine derivatives; and amine oxides, for example, (C₁₀-C₁₄)alkylamine oxides or N-acylaminopropylmorpholine oxides.

(iii) Amphoteric or Zwitterionic Surfactant(s):

The amphoteric or zwitterionic surfactants may be chosen from, for example, aliphatic secondary or tertiary amine derivatives in which the aliphatic radical chosen from linear and branched chais comprising 8 to 18 carbon atoms and comprising at least one water-solubilizing anionic group (for example carboxylate, sulfonate, sulfate, phosphate or phosphonate); (C₈-C₂₀)alkylbetaines; sulfobetaines; (C₈-C₂₀)alkylamido(C₁-C₆)alkylbetaines; and (C₈-C₂₀)alkylamido(C₁-C₆)alkylsulfobetaines.

The amphoteric or zwitterionic surfactants may be chosen from, for example, the products sold under the name Miranol, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures:
R₂—CONHCH₂CH₂—N(R₃)(R₄) (CH₂COO—)
wherein:
R₂ is chosen from alkyl radicals of an acid R₂—COOH present in hydrolysed coconut oil; heptyl radicals, nonyl radicals and undecyl radicals;
R₃ is a beta-hydroxyethyl group; and
R₄ is a carboxymethyl group; and
   R₂—CONHCH₂CH₂—N(B)(C)
wherein:
   B is —CH₂CH₂OX′;
   C is —(CH₂)ₙ—Y′, wherein z is chosen from 1 and 2;
   X′ is chosen from a —CH₂CH₂—COOH group and a hydrogen atom;
   Y′ is chosen from —COOH and —CH₂—CHOH—SO₃H radicals;
   R₂′ is chosen from alkyl radicals of an acid R₉-COOH present in coconut oil or in hydrolysed linseed oil; and alkyl radicals, for example, a C₇, C₉, C₁₁ or C₁₃ alkyl radical, a C₁₇ alkyl radical and its iso form, and unsaturated C₁₇ radicals.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Coco-amphodipropionate, Disodium Lauroamphopropionate, Disodium Caprylampho-dipropionate, Disodium Capryloamphodipropionate, Lauroamphodipropionic acid and Cocoamphodipropionic acid.

For example, the surfactant may be the cocoamphodiacetate sold under the trade name Miranol® C2M concentrate by the company Rhodia Chimie.

(iv) Cationic Surfactants:

The cationic surfactants may be chosen form, for example: salts of optionally polyoxyalkylenated primary, secondary or tertiary fatty amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; and amine oxides of cationic nature.

The amounts of surfactants present in the composition disclosed herein can range from 0.01% to 40%, for example, from 0.1% to 30% relative to the total weight of the composition.

The compositions disclosed herein may contain at least one nacreous agent or opacifier chosen from uncoated titanium oxides, coated titanium oxides, titanium micas, micas, fatty acid esters of polyols, and fatty ethers.

The titanium oxides that may be used in the compositions disclosed herein generally have a particle size ranging from 2 to 500 nanometers, further ranging from 2 to 300 nanometers and even further ranging from 2 to 50 nanometers.

The uncoated titanium oxides that may be used in the compositions disclosed herein may be chosen from, for example:
uncoated titanium oxides in powder form:
   Bayertitan and Titanium Dioxide A sold by the company Bayer,
   70110 Cardre UF TiO2 sold by the company Cardre,
uncoated titanium oxides as an aqueous dispersion containing 10%, 20% or 30% by weight of titanium oxide relative to the total weight of the aqueous dispersion, and with a particle size equal to 15, 20 or 60 nanometers:
Sunveil 1010, Sunveil 1020, Sunveil 1030, Sunveil 2020, Sunveil 2030, Sunveil 6010 and Sunveil 6030 sold by the company Catalysts & Chemicals,
Micro Titanium Dioxide-USP Grade sold by the company Color Techniques.

The coated titanium oxides that may be used in the compositions disclosed herein may be chosen form:
polydimethylsiloxane-coated titanium oxides (Cardre Ultrafine Titanium Oxide AS sold by the company Cardre),
polymethylhydrogenosiloxane-coated titanium oxides (polymethylhydrogenosiloxane-coated untreated titanium oxide sold under the trade name Cosmetic White SA-C47-051-10 by the company Miyoshi),
perfluoropolymethylisopropylether-coated titanium oxides (Cardre Mica FHC 70173 or 70170 Cardre UF T102 FHC sold by the company Cardre),
silica-coated titanium oxides (Spherititan AB sold by the company Catalysts & Chemicals),
Teflon-coated titanium oxides (CS-13997 Teflon Coated Titanium Dioxide sold by the company Clark Colors),
polyester-coated titanium oxides (Experimental Desoto Beads sold by the company Desoto),
chitosan-coated titanium oxides (CT-2 Titanium Dioxide MT-500SA sold by the company Dainihon Kasei),
N-lauryl-L-lysine-coated titanium oxides (LL-5 Titanium Dioxide A 100, LL-3 Titanium Dioxide MT-100SA or LL-5 Titanium Dioxide MT-500SA sold by the company Dainihon Kasei).

Among the titanium micas that may be used, for example in the compositions disclosed herein are the following products:
Flonac FS 20 C, Flonac ME 10 C, Flonac MG 10 C, Flonac MI 10 C, Flonac ML 10 C and Flonac MS 10 C sold by the company Eckart,
Timica Iridescent Red or Mattina Green sold by the company Engelhard,
Timiron Green MP-165 (17212), Timiron Starluster MP-115 (17200) or Timiron Super Sparkle MP-148 (17297) sold by the company Merck.

As micas that may be used, for example, in the compositions disclosed herein, mention may be made of Cosmetic Mica 280 BC from Whittaker Clark D and Mearlmica MMSV from Engelhard.

As fatty acid esters of polyols that may be used in the compositions disclosed herein, mention may be made, for example, of monostearates and distearates of ethylene glycol or of glycerol.

As fatty ethers that may be used in the compositions disclosed herein, mention may be made, for exmeple, of distearyl ether and hydroxystearyl cetyl ether.

The concentration of nacreous agent(s) or opacifier(s) that may be used for the purposes of composition disclosed herein may range from 0.05% to 10%, such as from 0.1% to 5% by weight relative to the total weight of the composition.

Disclosed herein is also a cosmetic process for shaping or holding the hair, comprising the use of the composition disclosed herein.

Also disclosed herein is methods of using the composition disclosed herein in a formulation chosen from hair lotions, hair gels, hair mousses, hair creams and hair lacquers for shaping or holding the hair.

The compositions disclosed herein may be used, for example, in pump-dispenser bottles or in aerosols. These containers may be one-compartment containers and use propellants selected from liquefied or compressed gases such as nitrogen, dimethyl ether or hydroxycarbons. They may also be two-compartment containers of the air-propelled bag aerosols type.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples which follow illustrate the invention without limiting its scope.

The cationic polyurethane of elastic nature used in the examples that follow is the polyurethane PU1 having an N-methyldiethanolamine/poly(tetramethylene oxide) molar ratio equal to 2, described in Patent Application FR 2 815 350.

### EXAMPLE 1

The present inventors prepared a composition according to the invention in the form of a styling lotion:

| Component | Weight % | |
|---|---|---|
| Cationic polyurethane of elastic nature | 2% | AM |
| Gaffix VC 713 (ISP) | 0.5% | AM |
| Ethanol | 5% | AM |
| Demineralized water | qs 100% | |

### EXAMPLE 2

The present inventors prepared a composition according to the invention in the form of a styling gel:

| Component | Weight % | |
|---|---|---|
| Elastic cationic polyurethane | 4% | AM |
| Jaguar HP105 | 1% | AM |
| Demineralized water | qs 100% | |

### EXAMPLE 3

The present inventors prepared a composition according to the invention in the form of a styling mousse:

| Component | Weight % |
|---|---|
| Elastic cationic polyurethane | 2% AM |
| Oxyethylenated (20 EO) sorbitan monolaurate | 0.2% AM |
| Demineralized water | 92.8 |
| Isobutane/propane (85/15) | 5% AM |

### EXAMPLE 4

The present inventors prepared a composition according to the invention in the form of a fixing spray in an air-propelled bag container. The fluid contains:

| Component | Weight % |
|---|---|
| Elastic cationic polyurethane | 2% AM |
| Demineralized water | qs 100% |

1. A styling cosmetic composition comprising, in a cosmetically acceptable medium comprising more than 50% by weight of water relative to the total weight of the composition, at least one cationic polyurethane of elastic nature.

2. A composition according to claim 1, wherein the cosmetically acceptable medium comprises more than 70% by weight of water relative to the total weight of the composition.

3. A composition according to claim 1, wherein the cosmetically acceptable medium comprises more than 85% by weight of water relative to the total weight of the composition.

4. A composition according to claim 1, wherein the at least one cationic polyurethane of elastic nature comprises:
(a1) cationic units derived from at least one tertiary or quaternary amine comprising at least two reactive functions containing labile hydrogen,
(a2) nonionic units derived from nonionic polymers bearing at their ends reactive functions comprising labile hydrogen and having a glass transition temperature (Tg), measured by differential thermal analysis, of less than 10° C., and
(a3) optionally, nonionic units derived from nonionic monomer compounds comprising at least two functions containing labile hydrogen, and
(b) units derived from at least one diisocyanate.

5. A composition according to claim 4, wherein the nonionic polymers forming the nonionic units (a2) have a glass transition temperature, measured by differential thermal analysis, of less than 0° C.

6. A composition according to claim 4, wherein the nonionic polymers forming the nonionic units (a2) have a glass transition temperature, measured by differential thermal analysis, of less than −10° C.

7. A composition according to claim 4, wherein the at least one cationic polyurethane of elastic nature has at least two different glass transition temperatures (T_{g}), wherein at least one of the glass transition temperatures is less than 10° C. and at least one other is greater than or equal to 20° C.

8. A composition according to claim 4, wherein the at least one cationic polyurethane of elastic nature has an immediate recovery ranging from 5% to 95% by weight.

9. A composition according to claim 4, wherein the at least one cationic polyurethane of elastic nature has an immediate recovery ranging from 20% to 90% by weight.

10. A composition according to claim 4, wherein the at least one cationic polyurethane of elastic nature has an immediate recovery ranging from 35% to 85% by weight.

11. A composition according to claim 4, wherein the cationic units (a1) are derived from at least one tertiary or quaternary amine containing two reactive functions containing labile hydrogen.

12. A composition according to claim 4, wherein the cationic units (a1) are derived from an amine chosen from: 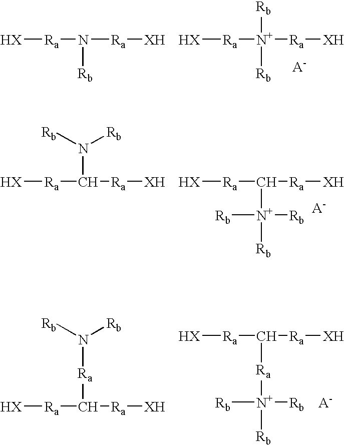
wherein
each Rₐ is independently chosen from linear and branched C₁₋₆ alkylene groups, C₃₋₆ cycloalkylene groups and arylene groups, optionally substituted with at least one halogen atom, wherein each Ra may comprise at least one hetero atom chosen from O, N, P and S;
each R_{b} is independently chosen from C₁₋₆ alkyls, C₃₋₆ cycloalkyls and aryl groups, optionally substituted with at least one halogen atom, wherein each Rb may comprise at least one hetero atom chosen from O, N, P and S;
each X is independently chosen from at least one oxygen and sulfur atom, an NH group, and an NR_{c} group, in which R_{c} comprises a C₁₋₆ alkyl group; and
A—is a physiologically acceptable counterion.

13. A composition according to claim 12, wherein the cationic units (a1) are chosen from N-methyldiethanolamine and N-tert-butyidiethanolamine derivatives.

14. A composition according to claim 11, wherein the units (a1) are derived from polymers containing tertiary and/or quaternary amine functions, bearing at their ends reactive functions containing labile hydrogen chosen from —OH, —NH₂, —NRc and —SH, and having a weight-average molecular mass ranging from 400 to 10 000, wherein R_{c} comprises a C₁₋₆ alkyl group.

15. A composition according to claim 4, wherein the polymers forming the nonionic units (a2) are chosen from polyethers, polyesters, polysiloxanes, copolymers of ethylene and of butylene, polycarbonates and fluoro polymers.

16. A composition according to claim 4, wherein the polymers forming the nonionic units (a2) have a weight-average molar mass ranging from 400 to 10 000.

17. A composition according to claim 4, wherein the polymers forming the nonionic units (a2) have a weight-average molar mass ranging from 1000 to 5000.

18. A composition according to claim 15, wherein the nonionic units (a2) are derived from poly(tetra methylene oxide).

19. A composition according to claim 4, wherein the units (b) are derived from diisocyanates chosen from methylenediphenyl diisocyanate, methylenecyclohexane diisocyanate, isophorone diisocyanate, toluene diisocyanate, naphthalene diisocyanate, butane diisocyanate and hexyl diisocyanate.

20. A composition according to claim 4, wherein the nonionic compounds forming the nonionic monomer units (a3) are chosen from neopentyl glycol, hexaethylene glycol and aminoethanol.

21. A composition according to claim 4, wherein the units (a1) are present in an amount ranging from 1% to 90% by weight, the units (a2) are present in an amount ranging from 10% to 80% by weight, and the units (a3) are present in an amount ranging from 0 to 50% by weight, relative to the total weight of the polymer, the units (b) being present in an essentially stoichiometric amount relative to the sum of the units (a1), (a2) and (a3).

22. A composition according to claim 4, wherein the units (a1) are present in an amount ranging from 5% to 60% by weight, the units (a2) are present in an amount ranging from 40% to 70% by weight, and the units (a3) are present in an amount ranging from 0 to 30% by weight, relative to the total weight of the polymer, the units (b) being present in an essentially stoichiometric amount relative to the sum of the units (a1), (a2) and (a3).

23. A composition according to claim 1, further comprising at least one fixing polymer.

24. A composition according to claim 23, wherein the concentration of the at least one fixing polymer ranges from 0.05% to 10% by weight relative to the total weight of the composition.

25. A composition according to claim 23, wherein the concentration of the at least one fixing polymer ranges from 0.1% to 5% by weight relative to the total weight of the composition.

26. A composition according to claim 23, wherein the concentration of the at least one additional fixing polymer ranges from 0.2% to 3% by weight relative to the total weight of the composition.

27. A composition according to claim 1, further comprising at least one at least one thickening polymer.

28. A composition according to claim 27, wherein the at least one thickening polymer is an associative thickening polymer.

29. A composition according to claim 27, wherein the concentration of the at least one thickening polymer ranges from 0.01% to 10% by weight relative to the total weight of the composition.

30. A composition according to claim 27, wherein the concentration of the at least one thickening polymer ranges from 0.1% to 5% by weight relative to the total weight of the composition.

31. A composition according claim 1, further comprising at least one surfactant.

32. A composition according to claim 31, wherein the at least one surfactant concentration ranges from 0.01% to 40% by weight relative to the total weight of the composition.

33. A composition according to claim 31, wherein the at least one surfactant concentration ranges from 0.1% to 30% by weight relative to the total weight of the composition.

34. A composition according to claim 1, further comprising at least one at least one nacreous agent or opacifier.

35. A composition according to claim 34, wherein the concentration of the at least one nacreous agent or opacifier ranges from 0.05% to 10% by weight relative to the total weight of the composition.

36. A composition according to claim 34, wherein the concentration of the at least one nacreous agent or opacifier ranges from 0.1% to 5% by weight relative to the total weight of the composition.

37. A cosmetic treatment process for shaping or holding the hairstyle, comprising applying to the hair, a styling cosmetic composition comprising, in a cosmetically acceptable medium comprising more than 50% by weight of water relative to the total weight of the composition, at least one cationic polyurethane of elastic nature.

38. A method of shaping or holding the hair comprising applying to the hair, a styling cosmetic composition comprising, in a cosmetically acceptable medium comprising more than 50% by weight of water relative to the total weight of the composition, at least one cationic polyurethane of elastic nature, wherein said method results in hair that exhibits at least one property chosen from hair holding that is persistent over time and hair shaping that is resistant to moisture.
